(19) United States
(12) Patent Application Publication
(10) Pub. No.: US 2020/0236974 A1
(43) Pub. Date: Jul. 30, 2020

(54) USE OF INOCULANTS AND ENZYMES TO INCREASE NUTRIENT RELEASE IN ANIMAL DIETS

(71) Applicant: DUPONT NUTRITION BIOSCIENCES APS, Copenhagen K (DK)

(72) Inventors: Shukun Yu, Malmo (SE); Karsten Matthias Kragh, Hoejbjerg (DK)

(21) Appl. No.: 16/637,760

(22) PCT Filed: Jul. 18, 2018
(86) PCT No.: PCT/US18/42581

§ 371 Date: Feb. 9, 2020

Related U.S. Application Data
(60) Provisional application No. 62/545,062, filed on Aug. 14, 2017.

(51) Int. Cl. A23K 50/10; A23K 10/12; C12N 9/26; C12N 9/34

(52) U.S. Cl. CPC A23K 50/10; A23K 10/12; C12Y 302/01001; C12N 9/2428; C12Y 302/01003; C12N 9/2414

(57) ABSTRACT

Disclosed are methods for improving the digestibility of high-moisture grain feed and/or rehydrated grain feed for animals which comprises a) processing the grain feed into fragments and b) contacting the grain feed fragments of step (a) with at least one starch hydrolase which is stable and active at a pH less than 5.0, optionally at least one protease, in combination with at least one inoculant comprising at least one bacterial strain.

### FIELD

The field relates to animal nutrition and, in particular, to the use of inoculants and enzymes to increase nutrient release in animal diets.

### BACKGROUND

Microbes can be used to improve the utilization of feed ingredients. For example, microbes are widely used as probiotics (also called direct fed microbials) for human health and animal nutrition. When such microbes are used to improve the utilization of feed ingredients, for example, to pre-treat silage, they are called “inoculants.” Silage inoculants are additives containing anaerobic lactic acid bacteria that are used to manipulate and enhance fermentation. Benefits include reduced fermentation loss of the silage and enhanced animal performance.

The most common lactic acid bacteria in commercial inoculants are Lactobacillus plantarum, Enterococcus faecium, various Pediococcus species and other Lactobacillus species.

Another option is to use enzymes to increase feed digestibility. For example, enzymes including phytase, xylanase, beta-glucanase and protease have also been tested for increasing the soluble nutrient levels by pre-incubation with feed components under anaerobic conditions (Ton Nu et al., High-moisture airtight storage of barley and triticale: Effect of moisture level and grain processing on nitrogen and phosphorus solubility. Animal Feed Science and Technology 210 (2015) 125-137). Treatment of corn silage with alpha-amylase has also been tested (Leahy et al., Effects of treating corn silage with alpha-amylase and (or) sorbic acid on beef cattle growth and carcass characteristics. J. Anim. Sci. 1990, 68:490-497). Cellulase and inoculant have been tested in alfalfa silage (Nadeau et al., Intake, digestibility, and composition of orchard grass and alfalfa silages treated with cellulase, inoculant, and formic acid fed to lambs. J. Anim. Sci. 2000. 78:2980-2989)

While silage inoculants or enzymes have been helpful in improving nutrient utilization of animal feed, there still is room for improvement. It has been found that a combination of at least one starch hydrolase alone or in combination with at least one protease and at least one inoculant can improve the nutrient utilization of animal feed.

### SUMMARY

In one embodiment, there is described a method for improving the digestibility of high-moisture grain feed and/or rehydrated grain feed for animals which comprises a) processing the grain feed into grain feed fragments and b) contacting the grain feed fragments of step (a) with at least one starch hydrolase that is stable and active at a pH less than 5.0 in combination with at least one inoculant comprising at least one bacterial strain.

In a second embodiment of claim 1 wherein, this starch hydrolase preferably has a starch binding domain that make it capable of hydrolyzing raw starch. Furthermore, the starch hydrolase is selected from the glycoside hydrolase family 13 and/or 15.

In a third embodiment, the starch hydrolase is selected from the group consisting of at least one alpha amylase or at least one glucoamylase.

In a fourth embodiment, the method described herein, further comprises at least one protease in step (b).

In a fifth embodiment, the protease is an endopeptidase and this endopeptidase is selected from the group consisting of metallopeptidases, serine proteases, threonine proteases and aspartic proteases.

In a sixth embodiment, the at least one inoculant comprises at least one lactobacillus strain.

In a seventh embodiment, the grain feed is selected from the group consisting corn silage, corn grain, barley silage, barley grain, sorghum, sorghum silage, oilseeds or a combination thereof.

In an eighth embodiment, the animal is a ruminant.

### BRIEF DESCRIPTION OF THE FIGURES

FIG. 1 depicts corn kernels broken by using a Buehler Mill.

FIG. 2 depicts the release of soluble nutrient of glucose by the interaction

Lactobacillus containing inoculant and enzymes from moisture corn.

### DETAILED DESCRIPTION

All patents, patent applications, and publications cited are incorporated herein by reference in their entirety.

In this disclosure, a number of terms and abbreviations are used. The following definitions apply unless specifically stated otherwise.

The articles “a”, “an”, and “the” preceding an element or component are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

The term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein in connection with a numerical value, the term “about” refers to a range of +1-0.5 of the numerical value, unless the term is otherwise specifically defined in context. For instance, the phrase a “pH value of about 6” refers to pH values of from 5.5 to 6.5, unless the pH value is specifically defined otherwise.

It is intended that every maximum numerical limitation given throughout this Specification includes every lower numerical limitation, as if such lower numerical limitations were expressly written herein. Every minimum numerical limitation given throughout this Specification will include every higher numerical limitation, as if such higher numerical limitations were expressly written herein. Every numerical range given throughout this Specification will include every narrower numerical range that falls within such broader numerical range, as if such narrower numerical ranges were all expressly written herein.

The term “glycoside hydrolase” is used interchangeably with “glycosidases”, “glycosyl hydrolases” and “starch hydrolases” Glycoside hydrolases assist in the hydrolysis of glycosidic bonds in complex sugar polymers (polysaccharides). Together with glycosyltransferases, glycosidases form the major catalytic machinery for the synthesis and breakage of glycosidic bonds. Glycoside hydrolases are classified into EC 3.2.1 as enzymes catalyzing the hydrolysis of O- or S-glycosides. Glycoside hydrolases can also be classified according to the stereochemical outcome of the hydrolysis reaction: thus, they can be classified as either retaining or inverting enzymes. Glycoside hydrolases can also be classified as exo or endo acting, dependent upon whether they act at the (usually non-reducing) end or in the middle, respectively, of an oligo/polysaccharide chain. Glycoside hydrolases may also be classified by sequence or structure based methods. They are typically named after the substrate that they act upon.

The term “starch” is used interchangeably with “amylum”. It is a polymeric carbohydrate consisting of a large number of glucose units joined by glycosidic bonds and is the most common storage carbohydrate in plants. Thus, “starch” can refer to any material comprised of the complex polysaccharide carbohydrates of plants, comprised of amylose and amylopectin with the formula (C₆H₁₀O₅)ₓ, wherein X can be any number. In particular, the term refers to any plant-based material including but not limited to grains, grasses, tubers and roots and more specifically wheat, barley, corn, rye, rice, sorghum, brans, cassava, millet, potato, sweet potato, and tapioca.

The terms “starch binding domain (SBD) or carbohydrate binding module (CBM)” are used interchangeably herein. SBDs can be divided into nine CBM families. As a source of energy, starch is degraded by many various amylolytic enzymes. However, only about 10% of them are capable of binding and degrading raw starch. These enzymes usually possess a distinct sequence-structural module called the starch-binding domain that mediates attachment to starch granules. SBD refers to an amino acid sequence that binds preferentially to a starch (polysaccharide) substrate or a maltosaccharide, alpha-, beta and gamma-cyclodextrin and the like. They are usually motifs of approximately 100 amino acid residues found in about 10% of microbial amylolytic enzymes.

The term “catalytic domain” refers to a structural region of a polypeptide which is distinct from the CBM and which contains the active site for substrate hydrolysis.

The terms “granular starch” and “raw starch” are used interchangeably herein and refer to raw (uncooked) starch, e.g., granular starch that has not been subject to gelatinization.

The term “alpha-amylase” is used interchangeably with alpha-1,4-D-glucan glucanohydrolase and glycogenase. Alpha-amylases (E.C. 3.2.1.1) usually, but not always, need calcium in order to function. These enzymes catalyze the endohydrolysis of alpha-1,4-glucosidic linkages in oligosaccharides and polysaccharides. Alpha-amylases act on, starch, glycogen, and related polysaccharides and oligosaccharides in a random manner, liberating reducing groups in the alpha-configuration.

The term “glucoamylase” (EC 3.2.1.3) is used interchangeably with glucan 1,4-alpha-glucosidase, amyloglucosidase, gamma-amylase, lysosomal alpha-glucosidase, acid maltase, exo-1,4-alpha-glucosidase, glucose amylase, gamma-1,4-glucan glucohydrolase, acid maltase, and 1,4-alpha-D-glucan hydrolase. This enzyme cleaves the last alpha-1,4-glycosidic linkages at the non-reducing end of amylose and amylopectin to yield glucose. It also cleaves the alpha-1,6-glycosidic linkages.

The term “protease” means a protein or polypeptide domain derived from a microorganism, e.g., a fungus, bacterium, or from a plant or animal, and that has the ability to catalyze cleavage of peptide bonds at one or more of various positions of a protein backbone (e.g., E.C. 3.4). The terms “protease”, “peptidase” and “proteinase” can be used interchangeably. Proteases can be found in animals, plants, fungi, bacteria, archaea and viruses. Proteolysis can be achieved by enzymes currently classified into six broad groups based on their catalytic mechanisms: aspartyl proteases, cysteine proteases, trypsin-like serine proteases, threonine proteases, glutamic proteases, and metalloproteases.

Peptidases can be classified by reaction catalyzed which is a functional classification or by molecular structure and homology which is a MEROPS classification.

TABLE 1
Functional classification:

| Peptidase | Type | Description | NC-IUBMB |
|---|---|---|---|
| Amino- | Exo | Cleaves one aa from N-terminus | EC 3.4.11 |
| Dipeptidyl- | Exo | Cleaves two aa from N-terminus | EC 3.4.14 |
| Tripeptidyl- | Exo | Cleaves three aa from N-terminus | EC 3.4.14 |
| Carboxyl- | Exo | Cleaves one aa from C-terminus | EC 3.4.16-18 |
| Peptidyldi- | Exo | Cleaves two aa from C-terminus | EC 3.4.15 |
| Di- | Exo | Cleaves dipeptides | EC 3.4.13 |
| Endo- | Endo | Cleaves internal peptide bonds | EC 3.4.21-25 |
| Oligo- | Endo | Endo-peptidase that only acts on | EC 3.4.21-25 |
| | | peptides. | |

TABLE 2
MEROPS classification

| Key amino acid residues and cofactors | |
|---|---|
| related to peptide bond breakage | MEROPS families |
| Serine | 45 |
| Cysteine | 65 |
| Metallo | 59 |
| Aspartic | 14 |
| Glutamic | 2 |
| Threonine | 4 |
| Unknown | 7 |
| Total | 196 |

The term “acid protease” means a protease having the ability to hydrolyze proteins under acidic conditions.

The terms “aspartic protease” and “aspartic acid protease” are used interchangeably herein and are a type of acid protease. Aspartic proteases (EC 3.4.23), also known as aspartyl proteases, an activated water molecule bound to one or more catalytic aspartate residues to hydrolyze a peptide bond in a polypeptide substrate. Generally, they have two highly conserved aspartates in the active site and are optimally active at acidic pH.

The abbreviation “AFP” refers to an aspartic fungal protease, that is, an aspartic protease from a fungal organism source.

The term “metalloprotease” is any protease whose catalytic mechanism involves a metal. Most metalloproteases require zinc, but some use cobalt. The metal ion is coordinated to the protein via three ligands. The ligands coordinating the metal ion can vary with histidine, glutamate, aspartate, lysine, and arginine. The fourth coordination position is taken up by a labile water molecule.

There are two subgroups of metalloproteinases include (a) exopeptidases, metalloexopeptidases (EC number: 3.4.17), and (b), metalloendopeptidases (3.4.24). Well known metalloendopeptidases include ADAM proteins and matrix metalloproteinases.

In the MEROPS database peptidase families are grouped by their catalytic type, the first character representing the catalytic type: A, aspartic; C, cysteine; G, glutamic acid; M, metallo; S, serine; T, threonine; and U, unknown. The serine, threonine and cysteine peptidases utilize the amino acid as a nucleophile and form an acyl intermediate—these peptidases can also readily act as transferases. In the case of aspartic, glutamic and metallopeptidases, the nucleophile is an activated water molecule. In many instances the structural protein fold that characterizes the clan or family may have lost its catalytic activity, yet retain its function in protein recognition and binding.

The term “serine protease” refers to enzymes that cleave peptide bonds in proteins, in which serine serves as the nucleophilic amino acid at the active site of the enzyme. Serine proteases fall into two broad categories based on their structure: the chymotrypsin-like (trypsin-like) and the subtilisins. In the MEROPS protease classification system, proteases are distributed among 16 superfamilies and numerous families. The family S8 includes the subtilisins and the family S1 includes the chymotrypsin-like (trypsin-like) enzymes. The subfamily S1E includes the trypsin-like serine proteases from Streptomyces organisms, such as Streptogricins A, B and C. The terms “serine protease”, “trypsin-like serine protease” and “chymotrypsin-like protease” are used interchangeably herein.

The term “threonine protease” refers a family of proteolytic enzymes having a threonine residue within the active site.

The terms “animal” and “subject” are used interchangeably herein. An animal includes all non-ruminant (including humans) and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai. Ruminants have the unique ability to convert roughage into protein and energy through their microbial/enzyme digestive systems. Accordingly, ruminants play an important role in the earth's ecology and in the food chain.

The primary difference between ruminants and nonruminants is that ruminants' stomachs have four compartments: the rumen, reticulum, omasum, and abomasum. In the first two chambers, the rumen and the reticulum, the food is mixed with saliva and separates into layers of solid and liquid material. Solids clump together to form the cud or bolus.

The cud is then regurgitated and chewed to completely mix it with saliva and to break down the particle size. Fiber, especially cellulose and hemicellulose, is primarily broken down in these chambers by microbes (mostly bacteria, as well as some protozoa, fungi and yeast) into the three major volatile fatty acids (VFAs): acetic acid, propionic acid, and butyric acid. Protein and nonstructural carbohydrate (pectin, sugars, and starches) are also fermented.

Though the rumen and reticulum have different names, they represent the same functional space as digesta and can move back and forth between them. Together, these chambers are called the reticulorumen. The degraded digesta, which is now in the lower liquid part of the reticulorumen, then passes into the next chamber, the omasum, where water and many of the inorganic mineral elements are absorbed into the blood stream.

After this, the digesta is moved to the true stomach, the abomasum. The abomasum is the direct equivalent of the monogastric stomach, and digesta is digested here in much the same way. Digesta is finally moved into the small intestine, where the digestion and absorption of nutrients occurs. Microbes produced in the reticulorumen are also digested in the small intestine. Fermentation continues in the large intestine in the same way as in the reticulorumen.

The term “fodder” as used herein refers to a type of animal feed, is any agricultural foodstuff used specifically to feed domesticated livestock, such as cattle, goats, sheep, horses, chickens and pigs. “Fodder” refers particularly to food given to the animals (including plants cut and carried to them), rather than that which they forage for themselves (called forage). Fodder is also called provender and includes hay, straw, silage, compressed and pelleted feeds, oils and mixed rations, and sprouted grains and legumes (such as bean sprouts, fresh malt, or spent malt). Most animal feed is from plants, but some manufacturers add ingredients to processed feeds that are of animal origin.

The term “feed” is used with reference to products that are fed to animals in the rearing of livestock. The terms “feed” and “animal feed” are used interchangeably.

The term “grain feed” as used herein refers to any grain used as feed for domestic livestock, such as cattle, poultry or other animals. In particular, grain feed refers to the seeds of plants which are typically feed to ruminant animals which may or may not include the outer hull, pod or husk of the seed. Examples include, but are not limited to, barley, corn, oats, sorghum, wheat (triticale), rye, and oilseeds such as soybean and rapeseed.

The term “high-moisture grain feed” refers to grain having at least 23% moisture. For example, “high-moisture corn” refers to corn harvested at 23 percent or greater moisture, stored and allowed to ferment in a silo or other storage structure, and used as feed for livestock.

The term “silage” refers to feed preserved by an anaerobic fermentation process (e.g. corn silage, hay silage, high-moisture corn, etc.) “Ensiled” refers to plant materials preserved by anaerobic fermentation and typically stored in a bag, bunker or upright silo.

“Oilseed” as used herein refers to any oil-containing seed, nut, kernel, or the like produced by a plant. All such plants, as well as their seeds, nuts, or kernels are contemplated for use herein. The oil content of small grains, e.g., wheat, is only 1-2%; that of oilseeds ranges from about 20% for soybeans to over 40% for sunflower and rapeseed (canola). The major world sources of edible seed oils are soybeans, sunflowers, rapeseed, cotton and peanuts. For example, the National Sustainable Agriculture Information Service lists the following as sources of oil for food, specialty, or industrial uses: almonds, apricot kernels, avocado, beech nut, bilberry, black currant, borage, brazil nut, calendula, caraway seed, cashew nut, castor seed, citrus seed, clove, cocoa, coffee, copra (dried coconut), coriander, corn seed, cotton seed, elderberry, evening primrose, grape seed, groundnut, hazelnut, hemp seed, jojoba, linseed, macadamia nut, mace, melon seed, mustard seed, neem seed, niger seed, nutmeg, palm kernel, passion fruit, pecan, pistachio, poppy seed, pumpkin seed, rape seed, raspberry seed, red pepper, rose hip, rubber seed, safflower seed, sea buckthorn, sesame seed, soybean, spurge, stinging nettle, sunflower seed, tropho plant, tomato seed, or walnut.

“Inoculants” contain bacteria selected to dominate the fermentation of the crops in the silo. Silage inoculants are divided in two categories depending on how they ferment a common plant sugar, glucose. Homofermenters produce just lactic acid and include some species of Lactobacillus like Lactobacillus plantarum, Pediococcus species, and Enterococcus species. The other category, heterofermenters, produce lactic acid, acetic acid or ethanol, and carbon dioxide. Lactobacillus buchneri is the best example of a heterofermenter.

As used herein, the term “functional assay” refers to an assay that provides an indication of a protein's activity. In some embodiments, the term refers to assay systems in which a protein is analyzed for its ability to function in its usual capacity. For example, in the case of a protease, a functional assay involves determining the effectiveness of the protease to hydrolyze a proteinaceous substrate.

Enzymes increase digestibility of modern animal feeds, which improve feed: grain ratios for ruminants and monogastric animals alike. Enzymes like cellulase and hemicellulase improve the nutritive value of silage and corn/soy based feeds. Other enzymes like alpha-galactosidase increase the nutritional value of Non-Starch Polysaccharides (NSP). Enzymes may benefit dogs and cats, improving the digestibility of pet foods and strengthening the immune system.

In one embodiment, there is described a method for improving the digestibility of high-moisture grain feed and/or rehydrated grain feed for animals which comprises a) processing the grain feed into grain feed fragments and b) contacting the grain feed fragments of step (a) with at least one starch hydrolase in combination with at least one inoculant comprising at least one bacterial strain.

Grain feed can be processed into fragments using any means known to those skilled in the art.

The grains in most of today's feeds are processed in some manner before being fed. Although some grains can be fed whole, processing, even if it is only grinding, usually makes the nutrients more available to the animal, thus improving digestibility and feed efficiency.

The primary goal of grain processing is to increase energy (starch) availability to improve animal performance. Typical processing methods reduce grain particle size with or without addition of water or steam. Some common grain processing methods are steam-flaking, dry-rolling, high-moisture harvesting and storage, and reconstitution (rehydration).

Commonly used grain processing methods include, but are not limited to, mechanical means such as grinding, cracking, rolling and crimping or thermal processing.

Grinding is done using either a hammermill or roller mill. Hammermills grind primarily by the impact of free-swinging hammers on the grain as it falls through the grinding chamber. Screens with specifically sized holes surround the grinding chamber and as the grain particles become small enough, they pass out through the holes. Roller mills have pairs of rolls, often two or three pairs per mill, that crush the grain as it passes between the rolls. The space between rolls can be adjusted to give various particle sizes.

Exemplary grain feed includes, but is not limited to, corn silage, corn grain, barley silage, barley grain, sorghum, sorghum silage, oilseeds or a combination thereof.

It has been found that when such fragmented grain feed is contacted with at least one starch hydrolase that is both stable and active at a pH less than 5.0 in combination with at least one inoculant comprising at least one bacterial strain the digestibility such feed is improved. Preferably, the starch hydrolase has a starch binding domain wherein said starch hydrolase is capable of hydrolyzing raw starch. Furthermore, the starch hydrolase is selected from the glycoside hydrolase family 13 and/or 15. Preferably, the starch hydrolase can be selected from the group consisting of alpha-amylases and glucoamylases.

A starch binding domain (is a structure motif possessed by many starch hydrolases including alpha amylases and glucoamylases (Christiansen et al., 2009, The carbohydrate-binding module family 20—diversity, structure, and function, FEBS J. 276: 5006-5029). In a broader sense, an SBD may also be referred to as a carbohydrate binding module (CBM). This structure motif facilitates the hydrolysis of raw starch by the starch hydrolases (Janecek et al. 2011, Structural and evolutionary aspects of two families of non-catalytic domains present in starch and glycogen binding proteins from microbes, plants and animals. Enzyme Microb. Technol. 49: 429-440.

Glycoside hydrolase family GH13 is the major glycoside hydrolase family acting on substrates containing α-glucoside linkages.

The α-amylase family represents a clan GH-H of three glycoside hydrolase families GH13, GH70 and GH77. The GH13 family includes, but is not limited to α-amylase (EC 3.2.1.1); oligo-1,6-glucosidase (EC 3.2.1.10); α-glucosidase (EC 3.2.1.20); pullulanase (EC 3.2.1.41); cyclomaltodextrinase (EC 3.2.1.54); maltotetraose-forming α-amylase (EC 3.2.1.60); isoamylase (EC 3.2.1.68); dextran glucosidase (EC 3.2.1.70); trehalose-6-phosphate hydrolase (EC 3.2.1.93); maltohexaose-forming α-amylase (EC 3.2.1.98); maltotriose-forming α-amylase (EC 3.2.1.116); maltogenic amylase (EC 3.2.1.133); neopullulanase (EC 3.2.1.135); malto-oligosyltrehalose trehalohydrolase (EC 3.2.1.141); limit dextrinase (EC 3.2.1.142); maltopentaose-forming α-amylase (EC 3.2.1.-); amylosucrase (EC 2.4.1.4); sucrose phosphorylase (EC 2.4.1.7); branching enzyme (EC 2.4.1.18); cyclomaltodextrin glucanotransferase (CGTase) (EC 2.4.1.19); 4-α-glucanotransferase (EC 2.4.1.25); isomaltulose synthase (EC 5.4.99.11); trehalose synthase (EC 5.4.99.16).

Glycoside hydrolase family 15 enzymes are exo-acting enzymes that hydrolyze the non-reducing end residues of α-glucosides. At present, the most commonly characterized activity is glucoamylase (EC 3.2.1.3), also known as amyloglucosidase, but glucodextranase (EC 3.2.1.70) and α,α-trehalose (EC 3.2.1.28) activities have been described. It has been found that fungal glucoamylases present some substrate flexibility and are able to degrade not only α-1,4-glycosidic bonds but also α-1,6-, α-1,3- and α-1,2-bonds to a lower degree.

Acidic stable and active alpha-amylases (EC 3.2.1.1) that can be used are selected from Glycoside Hydrolase Family GH13. There can be mentioned alpha-amylase from Aspergillus kawachii, A. clavatus. Furthermore, those alpha-amylases having granular starch hydrolyzing activity (GSH) or alpha-amylases that have been recombinantly engineered to have GSH activity can also be used. Such GSH activity is advantageous because these enzymes break down more of the starch, particularly any granular (raw) starch, which may be present in any feed containing molasses and the like. Alpha-amylases having GSH activity include, but are not limited to, alpha-amylases obtained from Aspergillus kawachi (e.g., AkAA), Aspergillus niger (e.g., AnAA), A. clavatus (AcAA), A. terreus (AtAA), and Trichoderma reesei (e.g., TrAA).

Alpha-amylases, AkAA, AcAA, and AtAA, have two carbohydrate binding domains, one of which belongs to carbohydrate binding module/domain family 20 (CBM20 or CD20) while the other is sometimes called a secondary binding site (SBS). SBSs and CBMs appear to function by 1) targeting the enzyme towards its substrate, 2) guiding the substrate into the active site groove, 3) substrate disruption, 4) enhancing processivity, 5) allosteric regulation, 6) passing on reaction products, and/or 7) anchoring to the cell wall of the parent microorganism.

Many of these putative functions agree with the functions ascribed to non-catalytic binding in CBMs. In contrast to CBMs, SBSs have a fixed position relative to the catalytic site, making them more or less suitable to take up specific functions (Cuyvers S., Dornez E., Delcour J. A., Courtin C. M. (2012), Occurrence and functional significance of secondary carbohydrate binding sites in glycoside hydrolases. Crit. Rev. Biotechnol. 32, 93-107).

Some commercially available alpha-amylases that may have GSH activity or enzymes used in carbohydrate hydrolysis processes are commercially available, see, e.g., TERMAMYL® 120-L, LC and SC SAN SUPER®, SUPRA®, and LIQUEZYME® SC available from Novo Nordisk A/S, FUELZYME® LF from Verenium, and CLARASE® L, SPEZYME® FRED, SPEZYME® XTRA, GC626, and GZYME® G997 available from Danisco, US, Inc., Genencor Division.

Glucoamylases (EC 3.2.1.3) are selected from Glycoside Hydrolase Family GH 15 and include, but are not limited to, glucoamylase from Trichoderma reesei (TrGA and its variant CS4, Brew1), glucoamylase from Aspergillus fumigatus (AfuGA), glucoamylase from Fusarium verticillioides (FvGA). Proteases (also called peptidases or proteinases) are enzymes capable of cleaving peptide bonds. Proteases have evolved multiple times, and different classes of proteases can perform the same reaction by completely different catalytic mechanisms. Proteases can be found in animals, plants, bacteria, archaea and viruses.

Proteolysis can be achieved by enzymes currently classified into six broad groups: aspartic proteases, cysteine proteases, serine proteases, threonine proteases, glutamic proteases, and metalloproteases.

Thus, in another embodiment, the method described herein can also include a protease along with the starch hydrolase and inoculant comprising at least one bacterial strain. Preferably, the protease is an endopeptidase selected from the group consisting of metallopeptidases, serine proteases, threonine proteases and aspartic proteases.

Preferably, the protease is an acid protease and, more preferably it is an acid fungal protease.

Any acid proteases can be used in this disclosure. For example, acid fungal proteases include those obtained from Aspergillus, Trichoderma, Mucor and Rhizopus, such as A. niger, A. awamori, A. oryzae, Trichoderma reesei, and M. miehei. AFP can be derived from heterologous or endogenous protein expression of bacteria, plants and fungi sources.

A metalloproteinase, or metalloprotease, is any protease enzyme whose catalytic mechanism involves a metal. Most metalloproteases require zinc, but some use cobalt. The metal ion is coordinated to the protein via three ligands.

There are two subgroups of metalloproteinases: (a) Exopeptidases, metalloexopeptidases (EC number: 3.4.17), and (b) Endopeptidases, metalloendopeptidases (3.4.24).

Well-known metalloendopeptidases include ADAM proteins and matrix metalloproteinases.

Serine proteases (or serine endopeptidases) are enzymes that cleave peptide bonds in proteins, in which serine serves as the nucleophilic amino acid at the (enzyme's) active site. They are found ubiquitously in both eukaryotes and prokaryotes. Serine proteases fall into two broad categories based on their structure: chymotrypsin-like (trypsin-like) or subtilisin-like.

Threonine proteases are a family of proteolytic enzymes harboring a threonine (Thr) residue within the active site.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin

Silage inoculants are forage additives containing lactic acid producing bacteria (LAB) and other anaerobic bacteria (such as Lactobacillus buchneri). These inoculants are used to manipulate and enhance fermentation in haylage (alfalfa, grass, cereal), corn silage and high-moisture corn. The goals are faster, more efficient fermentation with reduced fermentation losses, improved forage quality and palatability, longer bunk life, and improvements in animal performance.

Grain crops that are harvested for silage contain a natural population of both “good” and “bad” microbes. “Good” microbes include lactic acid producing bacteria (LAB) that help ensile the crop. “Bad” or spoilage microbes include clostridia, enterobacteria, bacilli, yeast and molds that negatively affect silage quality.

Spoilage microbes can cause poor fermentation, excessive dry matter, energy and nutrient losses, development of off flavors/aromas that reduce intakes and can even produce toxins that can compromise the health of animals.

Silage making relies on the conversion of plant sugars to acid. The acid decreases the pH and preserves the forage. The first step in the silage making process is to create oxygen-free (anaerobic) conditions through compacting and sealing the forage. Anaerobic (oxygen hating) bacteria are present in small numbers on all plant material. Once oxygen-free conditions have been achieved, these bacteria begin to multiply and convert plant sugars to fermentation acids. As fermentation acid levels increase, the pH drops preserving the forage as silage.

There are a variety of naturally occurring bacteria that can be present in silage. They produce a range of fermentation acids. A lactic fermentation is the most desirable because minimal energy is lost during the fermentation process and lactic acid produces palatable, high feed value silage.

The most common lactic acid producing bacteria (“LAB”) in commercial inoculants are Lactobacillus plantarum, Enterococcus faecium, various Pediococcus species and other Lactobacillus species. Species and specific strains of LAB in commercial inoculants have been selected because they grow rapidly and efficiently, and produce primarily lactic acid. They increase the fermentation rate, causing a more rapid decline in pH, with a slightly lower final pH. The products of fermentation are shifted, resulting in more lactic acid and less acetic acid, ethanol and carbon dioxide. Lactic acid is stronger than acetic acid, and contains almost as much energy as the original sugars.

Silage inoculants are mostly facultatively anaerobic such as LAB, which means they can grow whether or not oxygen is available. When oxygen is available inoculants help speed up the process of making silage material anaerobic. Once anaerobic conditions are achieved, these same bacteria switch to fast, efficient production of acids (lactic acid and some acetic acid) to reduce pH and prevent growth of spoilage microbes. When oxygen is less available, inoculants limit spoilage microbes that can grow in anaerobic conditions (e.g. clostridia, listeria).

Different bacterial strains vary in their ability to produce lactic acid. The most desirable strains are those that can convert sugar to lactic acid with minimal energy and dry matter loss. Any commercially available inoculants can be used. Examples of commercially available inoculants are Pioneer® brand inoculants Pioneer® brand 1132, 1127, 11H50 and 1174. There can also be mentioned Pioneer® brand 11C33 and 11CFT which contain a patented strain of Lactobacillus buchneri which reduces silage heating and spoilage at feed-out time.

Non-limiting examples of compositions and methods disclosed herein include:

1. A method for improving the digestibility of high-moisture grain feed and/or rehydrated grain feed for animals which comprises a) processing the grain feed into grain feed fragments and b) contacting the grain feed fragments of step (a) with at least one starch hydrolase that is both stable and active at a pH less than 5.0 in combination with at least one inoculant comprising at least one bacterial strain.

2. The method of embodiment 1 where in the starch hydrolase is selected from the glycoside hydrolase family 13 and/or 15.

3. The method of embodiment 1 wherein the starch hydrolase has a starch binding domain wherein said starch hydrolase is capable of hydrolyzing raw starch.

4. The method of embodiment 1, 2 or 3 wherein the starch hydrolase is selected from the group consisting of at least one alpha amylase or at least one glucoamylase.

5. The method of embodiment 1, 2, 3 or 4 wherein step (b) further comprises at least one protease.

6. The method of embodiment 5 wherein the protease is an endopeptidase.

7. The method of embodiment 6 wherein the endopeptidase is selected from the group consisting of metallopeptidases, serine proteases, threonine proteases and aspartic proteases.

8. The method of any of embodiments 1-7 wherein the at least one inoculant comprises at least one lactobacillus strain.

9. The method of any of embodiments 1-8 wherein grain feed is selected from the group consisting corn silage, corn grain, barley silage, barley grain, sorghum, sorghum silage, oilseeds or a combination thereof.

10. The method of embodiments 1-9 wherein the animal is a ruminant.

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used with this disclosure.

The disclosure is further defined in the following Examples. It should be understood that the Examples, while indicating certain embodiments, are given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1

### Materials Used in Subsequent Examples

The biological and protein samples listed on Table 1 where used in subsequent examples. Table 1 shows the enzyme type, source organism (when known) and internal or commercial source for samples, and patent references for sequences.

TABLE 1
List of enzymes, components and biomaterial evaluated.

| Protein or | | | |
|---|---|---|---|
| Product | Product | | |
| Name | Type | Organism Sources | References |
| LAT | alpha- | Bacillus licheniformis | Patent |
| | amylase | | US2013/ |
| | | | 0171296 |
| AkAA | alpha- | Recombinant, Aspergillus | U.S. Pat. No. |
| | amylase | kawachii source | 7,354,752 |
| AcAA | alpha- | Recombinant, Aspergillus | U.S. Pat. No. |
| | amylase | clavatus source | 8,945,889 |
| TrGA | glucoamylase | Recombinant, Trichoderma | U.S. Pat. No. |
| | | reseei source | 7,413,879 |
| CS4 | glucoamylase | Recombinant, variant, | U.S. Pat. No. |
| | | Trichoderma reseei source | 8,058,033 |
| Brew1 | glucoamylase | Recombinant, variant, | U.S. Pat. No. |
| | | Trichoderma reseei source | 8,809,023 |
| FvGA | glucoamylase | Recombinant, Fusarium | Patent |
| | | verticillioides source | WO2016100871 |
| AfuGA | glucoamylase | Recombinant, Aspergillus | Patent |
| | | fumigatus source | US20160115509 |
| AFP | protease | Recombinant, T. reseei | U.S. Pat. No. |
| | | source | 8,288,517 |
| Pioneer ® | Inoculants | Lactobacillus buchneri, L. | Pioneer, a |
| brand | | plantarum | DuPont |
| 11B91* | | | company |
| Pioneer ® | corn | Zea mays | Pioneer, a |
| brand | | | DuPont |
| P7524 | | | company |

According to the producer, Pioneer® Brand 11B91 is a high-moisture corn inoculant product designed to: Improve fermentation, retain nutrient content and enhance digestibility of ensiled high-moisture corn. Use in high-moisture corn ensiled at the proper maturity in upright, bunker or bag silos at moistures ranging from 22% to 32%. The protein concentration of the enzymes used are given below in Table 2.

### Example 2

### Hydrolysis of Broken Corn with Starch Hydrolases and their Combinations with Acidic Protease in the Pre-Treatment with Microbial Inoculants

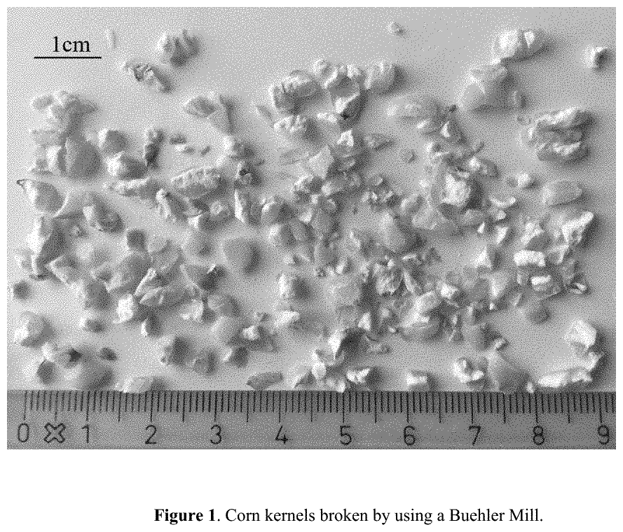

Pioneer corn kernels of cultivar P7524 were used. It consisted of 88.2% dry matter (DM), 9.3% crude protein (CP), 2.0% acid detergent fiber (ADF), 6.6% neutral detergent fiber treated with amylase (aNDF), 78.5% non-fibrous carbohydrates (NFC), 89.0% total digestible nutrients (TDN). It was first broken into about 3-10 fragments using Buehler Mill (Bühler AG, Uzwil, Switzerland) at setting 9. Fragments had a length between 1 mm and 0.9 cm (FIG. 1). Fragments smaller than 1 mm in diameter were removed by sieving. To 100 g of the milled corn placed in OBH Nordica food sealer plastic bags with volume of about 0.7 liter (OBH Nordica Group AB, Sundbyberg, Sweden), were added 26 g tap water so that the final moisture was 30% (w/w), 100 μL diluted inoculant (Pioneer 11B91) and enzymes as set forth in Table 2.

TABLE 2
The compositions of the enzyme plus inoculant incubation mixtures

| | | | | | | Enzyme | |
|---|---|---|---|---|---|---|---|
| | | | | | | protein | |
| | | | | | | dosed | |
| | | | | | Enzyme | in ppm | |
| | | | Inoculant | Enzyme | protein | based on | Number |
| | Corn | Water | 11B91 | (μL) | (mg) | corn dry | of |
| Treatments | [g] | [g] | [μL] | dosed | added | matter | repetitions |
| Time Zero (Blank— | 100 | 26 | | 0 | 0 | 0 | 3 |
| 20° C.) | | | | | | | |
| Control (inoculant | 100 | 26 | 100 | 0 | 0 | 0 | 5 |
| only) | | | | | | | |
| AFP + inoculant | 100 | 26 | 100 | 5 | 0.66 | 7.5 | 3 |
| LAT + inoculant | 100 | 26 | 100 | 5 | 1.02 | 11.6 | 3 |
| AkAA + inoculant | 100 | 26 | 100 | 5 | 0.585 | 6.6 | 3 |
| AcAA + inoculant | 100 | 26 | 100 | 5 | 0.475 | 5.4 | 3 |
| CS4 + inoculant | 100 | 26 | 100 | 5.6 | 0.595 | 6.8 | 3 |
| TrGA + inoculant | 100 | 26 | 100 | 5 | 0.285 | 3.2 | 3 |
| TrGA + inoculant | 100 | 26 | 100 | 11.6 | 0.661 | 7.5 | 2 |
| Brew1 + inoculant | 100 | 26 | 100 | 6.9 | 0.656 | 7.5 | 3 |
| AfuGA + inoculant | 100 | 26 | 100 | 7.8 | 0.663 | 7.5 | 3 |
| FvGA + inoculant | 100 | 26 | 100 | 4.6 | 0.851 | 9.6 | 3 |
| AkAA + TrGA + | 100 | 26 | 100 | 5 | 0.700 | 8 | 3 |
| AFP* + inoculant | | | | | | | |
| AcAA + CS4 + | 100 | 26 | 100 | 5 | 0.360 | 4.1 | 3 |
| AFP* + inoculant | | | | | | | |

*The protein weight ratio of the alpha-amylases, glucoamylases and fungal acidic protease of AkAA/AcAA, TrGA/CS4, and APF are 29%, 70% and 1%, respectively.

The Pioneer 11B91 inoculant was prepared by suspending 1 g of the powdery product in 1000 g of tap water and mixed well as the diluted inoculant. The plastic bags containing the fragmented corn, the inoculant with and without (control) and the enzymes to be tested were vacuum sealed using a vacuum sealer from OBH Nordica. These sealed bags were incubated at 22° C. or at −20° C. (blank) for 35 days.

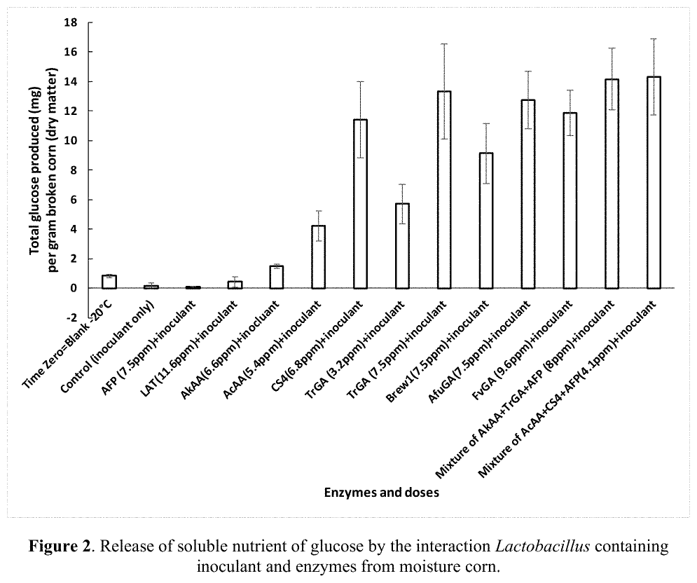

After a 35-day incubation at 22° C., 5 g of the corn fragment sample were taken from each of the sealed bags and was transferred to a 50 mL Falcon centrifuge tube to which was added 15 mL of MilliQ-water. The mixture was mixed for 1 minute and allowed to stand for 3 minutes. The supernatant (soluble nutrient extract) was collected by centrifugation at 3500 rpm for 10 min at 15° C. The supernatant was then filtered by passing through Millipore steriflip 0.22 μm (CAT#SCGP00525). The filtrate was measured for pH (Table 3). Glucose concentrations were quantified by HPLC as is shown in FIG. 2. For the HPLC quantification of glucose, the filtrate (40 μl) was injected to HPLC analysis on an Aminex HPX-87N HPLC column (Bio-Rad) at a flow rate of 0.6 ml/min, column oven temperature set at 75° C., over 15 min using water as eluent. Glucose peak were detected using an inline RI (refractory index) detector, and the peak areas were integrated using Chromeleon software (Dionex) according to the manufacturer's instructions and compared to the peak areas of glucose standards at 0, 0.025, 0.125, 0.25, 0.5, 1.0 and 2.0 mg/ml.

The results are set forth in Table 3 and show that the corn dosed with the inoculant 11B91 which is a mixture Lactobacillus buchneri and L. plantarum (according to the manufacturer) decreased the pH from 6.46 (the blank or the starting pH) to pH4.25 (Control).

It was observed that it may take up to 3 days of incubation to achieve a pH decrease of about 2.2 pH units using the inoculant 11B91 alone under the experimental conditions described herein.

TABLE 3
The pH of the water extract of the
pre-treated high-moisture corn after 35 days

| | Enzyme concentration | Number | |
|---|---|---|---|
| | (ppm based on corn | of | Final |
| Treatments | dry matter) | repetitions | pH |
| Time Zero (Blank −20° C.) | 0 | 3 | 6.46 |
| Control | 0 | 5 | 4.25 |
| AFP plus inoculant | 7.5 | 3 | 4.27 |
| LAT plus inoculant | 11.6 | 3 | 4.03 |
| AkAA plus inoculant | 6.6 | 3 | 3.97 |
| AcAA plus inoculant | 5.4 | 3 | 4.04 |
| CS4 plus inoculant | 6.8 | 3 | 4.02 |
| TrGA plus inoculant | 3.2 | 3 | 4.03 |
| TrGA plus inoculant | 7.5 | 2 | 4.03 |
| Brew1 plus inoculant | 7.5 | 3 | 4.03 |
| AfuGA plus inoculant | 7.5 | 3 | 4.05 |
| FvGA plus inoculant | 9.6 | 3 | 4.03 |
| AkAA + TrGA + AFP plus | 8.0 | 3 | 4.04 |
| inoculant | | | |
| AcAA + CS4 + AFP plus | 4.1 | 3 | 3.94 |
| inoculant | | | |

The blank is the pH of the high-moisture corn without an inoculant after 35 days of storage at −20° C. instead of 22° C. incubation. Ctrl is the control in which the high-moisture corn was incubated with the inoculant without the addition of enzymes. For the details of the enzymes added, see Table 2 above. The number (n) of repetitions is 2-5.

A pH4.25 and pH4.27 was measured for the control (inoculant only) and protease plus inoculant treatments respectively.

When starch hydrolase enzyme treatments were added to the protease and inoculant combination, a further decrease in pH of about 0.2 pH units (to pH around 4.0) was observed.

The further decrease of 0.2 pH units observed when both a starch hydrolase and protease were used in combination with an inoculant. This may be due to the increased availability of nutrients available to the Lactobacilli present which in turn leads to the production of more organic acids. Organic acids (known as an acidifier or an acidulant) are known to be important feed additives for the livestock industry.

FIG. 2 and Table 4 show that neither the control sample (inoculant alone) nor the AFP (protease and inoculant) and LAT (alpha amylase and inoculant) treated sample had a glucose amount greater than 0.5 mg per gram corn, in fact the glucose levels in these three treatments were even lower than in the blank due to the consumption of the free glucose found in corn by the inoculant.

LAT is a bacterial alpha-amylase that generates maltose, maltosaccharides and glucose. It is believed that glucose production was low due to incubation with the Lactobacillus inoculant that resulted in consumption of some of the glucose generated by LAT.

The addition of the acidic stable and active alpha-amylases AkAA, AcAA, glucoamylases TrGA, CS4, Brew1, AfuGA and FvGA in a dose of 3-12 ppm gave a glucose release of in the range of 1-14 mg per gram corn as is shown in Table 4. Specifically, the glucose released is in the range 0.1% to 1% of the fermented corn. The 3-enzyme mixture AcAA+CS4+AFP (alpha amylase, glucoamylase and protease plus inoculant) released a high amount of glucose based on the amount of enzyme dosed (see Table 4). The mixture of AkAA+TrGA+AFP generated the next highest amount of glucose based on the amount of enzyme dosed, followed by TrGA, then AfuGA and finally CS4. Among the glucoamylases tested, FvGA was found to be less efficient.

The data presented herein in Table 4 shows that the two acidic stable and acidic active alpha-amylases of AkAA and AcAA which that have a starch binding domain (SBD) are 3-6 times more efficient than an alpha-amylase such as LAT which lacks an SBD.

TABLE 4
The ratio between glucose released and enzyme protein dosed.

| | Enzyme | Glucose (mg produced per | Efficacy |
|---|---|---|---|
| | dose | gram high-moisture corn | Glucose(mg)/ |
| Treatment | (ppm) | based on dry matter) | enzyme (ppm) |
| Blank | 0 | 0.83 | |
| Ctrl | 0 | 0.17 | |
| AFP plus inoculant | 7.5 | 0.08 | 0.01 |
| LAT plus inoculant | 11.6 | 0.42 | 0.04 |
| AkAA plus inoculant | 6.6 | 1.47 | 0.22 |
| AcAA | 5.4 | 4.24 | 0.78 |
| CS4 plus inoculant | 6.8 | 11.40 | 1.68 |
| TrGA plus inoculant | 3.2 | 5.72 | 1.79 |
| TrGA plus inoculant | 7.5 | 13.32 | 1.78 |
| Brew1 plus inoculant | 7.5 | 9.12 | 1.22 |
| AfuGA plus inoculant | 7.5 | 12.73 | 1.70 |
| FvGA plus inoculant | 9.6 | 11.86 | 1.24 |
| AkAA + TrGA + AFP | 8 | 14.16 | 1.77 |
| plus inoculant | | | |
| AcAA + CS4 + AFP | 4.1 | 14.29 | 3.48 |
| plus inoculant | | | |

What is claimed is:

1. A method for improving the digestibility of high-moisture grain feed and/or rehydrated grain feed for animals which comprises a) processing the grain feed into grain feed fragments and b) contacting the grain feed fragments of step (a) with at least one starch hydrolase that is both stable and active at a pH less than 5.0 in combination with at least one inoculant comprising at least one bacterial strain.

2. The method of claim 1 wherein the starch hydrolase has a starch binding domain wherein said starch hydrolase is capable of hydrolyzing raw starch.

3. The method of claim 1 where in the starch hydrolase is selected from the glycoside hydrolase family 13 and/or 15.

4. The method of claim 1, 2 or 3 wherein the starch hydrolase is selected from the group consisting of at least one alpha amylase or at least one glucoamylase.

5. The method of claim 1, 2, or 3 wherein step (b) further comprises a protease.

6. The method of claim 4 wherein step (b) further comprises a protease.

7. The method of claim 4 or 6 wherein the protease is an endopeptidase.

8. The method of claim 5 wherein the protease is an endopeptidase.

9. The method of claim 6 or 8 wherein the endopeptidase is selected from the group consisting of metallopeptidases, serine proteases, threonine proteases and aspartic proteases.

10. The method of claim 7 wherein the endopeptidase is selected from the group consisting of metallopeptidases, serine proteases, threonine proteases and aspartic proteases.

11. The method of claim of claim 1, 2, or 3 wherein the at least one inoculant comprises at least one lactobacillus strain.

12. The method of claim of claim 4 wherein the at least one inoculant comprises at least one lactobacillus strain.

13. The method of claim of claim 5 wherein the at least one inoculant comprises at least one lactobacillus strain

14. The method of claim of claim 6, or 8 or 10 wherein the at least one inoculant comprises at least one lactobacillus strain.

15. The method of claim of claim 7 wherein the at least one inoculant comprises at least one lactobacillus strain

16. The method of claim of claim 9 wherein the at least one inoculant comprises at least one lactobacillus strain

17. The method of claim 1 wherein grain feed is selected from the group consisting corn silage, corn grain, barley silage, barley grain, sorghum, sorghum silage, oilseeds or a combination thereof.

18. The method of claim 1 wherein the animal is a ruminant.
